(19) United States
(12) Patent Application Publication
(10) Pub. No.: US 2019/0374157 A1
(43) Pub. Date: Dec. 12, 2019

(54) METHODS AND APPARATUS FOR PROVIDING PERSONALIZED BIOFEEDBACK FOR THE TREATMENT OF PANIC ATTACKS

(71) Applicants: Ryan McGinnis, Shelburne, VT (US); Ellen McGinnis, Shelburne, VT (US)

(72) Inventors: Ryan McGinnis, Shelburne, VT (US); Ellen McGinnis, Shelburne, VT (US)

(21) Appl. No.: 16/432,997

(22) Filed: Jun. 6, 2019

Related U.S. Application Data
(60) Provisional application No. 62/681,926, filed on Jun. 7, 2018.

(51) Int. Cl. A61B 5/00; G16H 10/60; G16H 20/70

(52) U.S. Cl. CPC A61B 5/486; A61B 5/0022; A61B 5/1118; G16H 20/70; A61B 5/6898; G16H 10/60

(57) ABSTRACT

Methods and apparatus for providing personalized biofeedback therapy to a user experiencing a panic attack are described. The method comprises receiving an indication that the user is experiencing a panic attack, receiving, from a recommendation system, a recommended biofeedback therapy for the user, providing the recommended biofeedback therapy to the user during the panic attack, and transmitting to the recommendation system, information related to the panic attack after the panic attack has subsided, wherein the information related to the panic attack is used to provide an updated recommended biofeedback therapy for the user.

### RELATED APPLICATIONS

This application claims priority under 35 U.S.C. § 119(e) to U.S. Provisional Application No. 62/681,926, filed Jun. 7, 2018 and titled, “METHODS AND APPARATUS FOR PROVIDING PERSONALIZED BIOFEEDBACK FOR THE TREATMENT OF PANIC ATTACKS,” the entire contents of which is incorporated by reference herein.

### BACKGROUND

Over 11% of adults, or nearly 27.5 million Americans, have had a panic attack in the last 12 months. However, only 16% of panic attack sufferers seek treatment for their attacks. Left untreated, panic attacks predict the onset of mental illnesses including social phobia, specific phobia, generalized anxiety disorder, depression, and substance use disorders as well as greater persistence, co-morbidity, and functional impairment of mental disorders. Conventional techniques for treating panic attacks include receiving psychological (e.g., cognitive behavioral therapy) or pharmacological intervention.

### SUMMARY

According to one aspect of the technology described herein, some embodiments are directed to a mobile electronic device configured to provide personalized biofeedback therapy to a user experiencing a panic attack. The mobile electronic device comprises at least one computer processor, and at least one storage device encoded with a plurality of computer-executable instructions that, when executed by the at least one computer processor perform a method. The method comprises receiving an indication that the user is experiencing a panic attack, receiving, from a recommendation system, a recommended biofeedback therapy for the user, providing the recommended biofeedback therapy to the user during the panic attack, and transmitting to the recommendation system, information related to the panic attack after the panic attack has subsided, wherein the information related to the panic attack is used to provide an updated recommended biofeedback therapy for the user.

According to another aspect of the technology described herein, some embodiments are directed to a computer system, comprising at least one computer processor, and at least one storage device encoded with a plurality of computer-executable instructions that, when executed by the at least one computer processor perform a method. The method comprises receiving a request to provide a biofeedback therapy recommendation for a user, determining based, at least in part, on data associated with the user and data associated with a plurality of other users for whom biofeedback therapy was previously provided, the biofeedback therapy recommendation, and transmitting the biofeedback therapy recommendation to a mobile application executing on a mobile device.

According to another aspect of the technology described herein, some embodiments are directed to a method of providing personalized biofeedback therapy to a user experiencing a panic attack. The method comprises receiving an indication that the user is experiencing a panic attack, receiving, from a recommendation system, a recommended biofeedback therapy for the user, providing the recommended biofeedback therapy to the user during the panic attack, and transmitting to the recommendation system, information related to the panic attack after the panic attack has subsided, wherein the information related to the panic attack is used to provide an updated recommended biofeedback therapy for the user.

It should be appreciated that all combinations of the foregoing concepts and additional concepts discussed in greater detail below (provided such concepts are not mutually inconsistent) are contemplated as being part of the inventive subject matter disclosed herein.

### BRIEF DESCRIPTION OF THE DRAWINGS

Various non-limiting embodiments of the technology will be described with reference to the following figures. It should be appreciated that the figures are not necessarily drawn to scale.

FIG. 1 is a block diagram of a system for providing personalized biofeedback therapy to a user in accordance with some embodiments;

FIG. 2A is a plot of a signal extracted from a mobile phone video of a user's fingertip that is used to estimate heart rate in accordance with some embodiments;

FIG. 2B is a spectrogram of the signal illustrated in FIG. 2A;

FIG. 2C is a plot of a power spectrum for determining an average heart rate value in accordance with some embodiments;

FIG. 3A is a plot of a signal extracted from an instantaneous heart rate measurement that is used to estimate respiratory rate in accordance with some embodiments;

FIG. 3B is a power spectrum of the signal illustrated in FIG. 3A.

FIG. 4 is a flowchart of a process for determining a personalized biofeedback therapy for a user in accordance with some embodiments;

FIG. 5 is a flowchart of a process for providing personalized biofeedback therapy to a user in accordance with some embodiments; and

FIG. 6 is a flowchart of a process for generating a biofeedback therapy recommendation for a user in accordance with some embodiments.

### DETAILED DESCRIPTION

Conventional techniques for treating patients predisposed to having panic attacks suffer from various limitations. For example, pharmacological interventions used to treat panic attacks, such as antidepressants or benzodiazepines, suffer from significant side effects. Benzodiazepines in particular act as sedatives to reduce the symptoms of an impending attack and do not aid in preventing future attacks. It has been reported that the most common psychological intervention used for treating panic attacks, cognitive behavioral therapy (CBT) with in-vivo exposure, resulted in only one third of patients having improved symptoms one year later. Moreover, many therapists are not trained in treating panic attacks, and often prescribe deep breathing exercises that have been shown to worsen the frequency and severity of attacks. The inventors have recognized and appreciated that conventional techniques for treating panic attacks may be improved by providing individualized treatment in a manner that is decoupled from the clinic to help people obtain access to treatment when they need it.

According to cognitive-behavioral theories of panic, emotional activation leads to hyperventilation, which increases blood pH. This increase leads to a cascade of uncomfortable somatic symptoms developing abruptly and peaking within ten minutes. Individuals experiencing these symptoms often feel helpless and scared, thus exacerbating their hyperventilation. When accessible and available, psychological intervention has been shown to be extremely effective for the treatment of panic attacks. In particular, biofeedback therapy, which provides the patient in vivo information about their physiological arousal (e.g., in the form of elevated respiratory and heart rates), has been shown to make significant improvements in 81% of patients who remain panic-free at two years follow-up. Biofeedback has been effective in helping individuals feel more in control of their bodily reactions and react less fearfully to them, thus ending the cycle of panic by acquiring a sense of mastery, and giving immediate relief from the longstanding feelings of helplessness. Studies demonstrate that face-to-face biofeedback sessions with a clinician are equivalent to self-exposure in patients suffering from panic attacks.

Although biofeedback therapy has been shown to decrease occurrences of panic attacks, use of the therapy has been limited to the clinic due, at least in part, to the need for specialized equipment to quantitatively track physiological parameters (e.g., heart and respiratory rate) during an attack. Some embodiments are directed to a portable system configured to provide biofeedback to a user during a panic attack using a mobile device that quantitatively tracks user physiological parameters (e.g., heart rate, respiratory rate) during an attack and provides personalized biofeedback to the user based on the tracked parameters. Successful measurement of physiological parameters in accordance with some embodiments enables biofeedback therapy to be provided to patients whenever and wherever their panic attacks occur.

The efficacy of biofeedback therapy for individual patients is often dependent on the method used to present the feedback to the patient. For example, some patients respond best to tactile feedback, other patients respond best to visual or audio feedback, and yet other patients respond best to feedback provided using a combination of modalities. In conventional clinic-based approaches to treating panic attacks, the determination of which type of biofeedback is likely to be most effective for a particular patient is made based on a clinician's observations of the patient's response to each of multiple forms of feedback. Realizing that access to a clinician during a panic attack is typically not possible, some embodiments are directed to techniques for assessing the effectiveness of biofeedback provided to a user during a panic attack that occurs outside of a clinical environment. In particular, the effectiveness of biofeedback may be assessed in some embodiments based, at least in part, on one or more physiological parameters (e.g., heart rate, respiratory rate) of the user tracked as the biofeedback is provided during a panic attack and/or information that the user provides to the system (e.g., by answering a series of questions) during or following resolution of the attack. Data recorded during and/or following a panic attack are provided as input to a recommender system, which automatically recommends biofeedback for the user for future panic attacks based on the updated information.

As described in more detail below, some embodiments include a mobile device (e.g., a mobile phone or wearable device), configured to administer biofeedback during a panic attack and capture the patient's response (e.g., in terms of measured respiratory rate and heart rate during a panic attack, long-term frequency and severity of attacks) to the biofeedback. The captured response to the biofeedback may be processed using artificial intelligence to automatically personalize the biofeedback approach to a given user thereby providing a closed-loop experience, where the system learns about the type of biofeedback that is most effective for the patient as more information is added to the system.

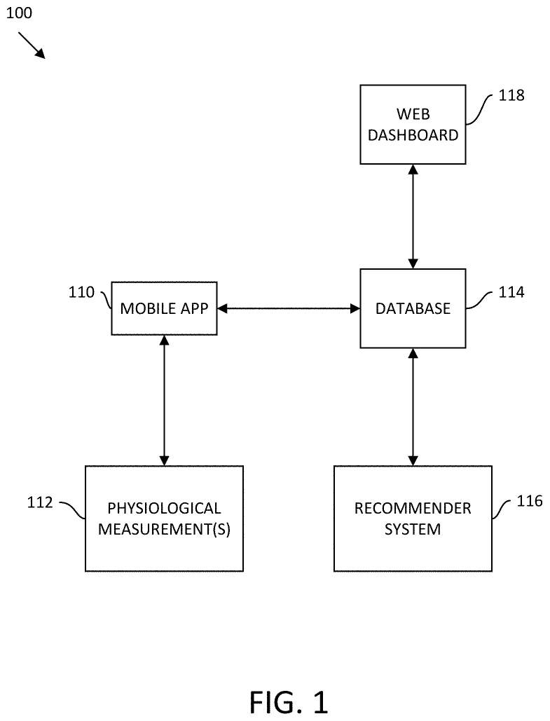

FIG. 1 schematically illustrates a system 100 configured to provide personalized biofeedback in accordance with some embodiments. As shown, system 100 include a mobile application 110 configured to be implemented using one or more processors of a mobile device (e.g., a smartphone and/or wearable device). Mobile application 110 is configured to present a user interface to a user of system 100 that provides biofeedback to the user during a panic attack. Characteristics of the biofeedback provided to the user are determined based on a personalized recommendation made by recommender system 116, as described in more detail below. Mobile application 110 facilitates communication among other components of system 100 including physiological measurement(s) component 112, database 114, and recommender system 116. System 100 also includes a web dashboard 118 configured to enable users or other stakeholders (e.g., physicians, health systems, insurance companies, etc.) to visualize user data.

Collectively, the components of system 100 enable mobile application 110 to provide one or more measurements of a user's physiological response to a user during a panic attack and to personalize a biofeedback therapy based on information about the patient's history of panic attacks and the history of users with similar characteristics, while also allowing interested stakeholders (e.g., the user, physician, employer, insurer, health system) to track progress and compliance.

As described briefly above, system 100 includes physiological measurements component 112 configured to record one or more physiological parameters, non-limiting examples of which include heart rate and respiratory rate. The recorded physiological parameters may be provided as input to recommender system 116, which uses the parameters to generate a biofeedback recommendation for a particular user. Physiological measurements component 112 may be implemented in any suitable way. In some embodiments, physiological measurements component 112 may be implemented, at least in part, using a device separate from a mobile device configured to implement mobile application 110. For example, a wearable device such as a heart rate monitor or smart watch may be configured to record raw physiological data and to provide the recorded physiological data to one or more processors programmed to derive physiological information from the raw physiological data in real-time. The data may be transmitted between the devices using any suitable wired or wireless technique, an example of which includes Bluetooth. In some embodiments the separate device used to record the raw physiological data may also derive physiological information used to provide biofeedback and the derived physiological information (rather than or in addition to the raw physiological data) may be provided to mobile application 110.

In yet further embodiments, physiological measurements component 112 may be integrated as part of the mobile device upon which mobile application 110 is executing, such that a separate device to record the raw physiological data is not necessary. Integrating the physiological measurements component 112 as part of the mobile device upon which mobile application 110 is executing may enable a simpler and faster system 100, by eliminating the need to transfer data between two different devices (e.g., a heart rate monitor and a mobile phone).

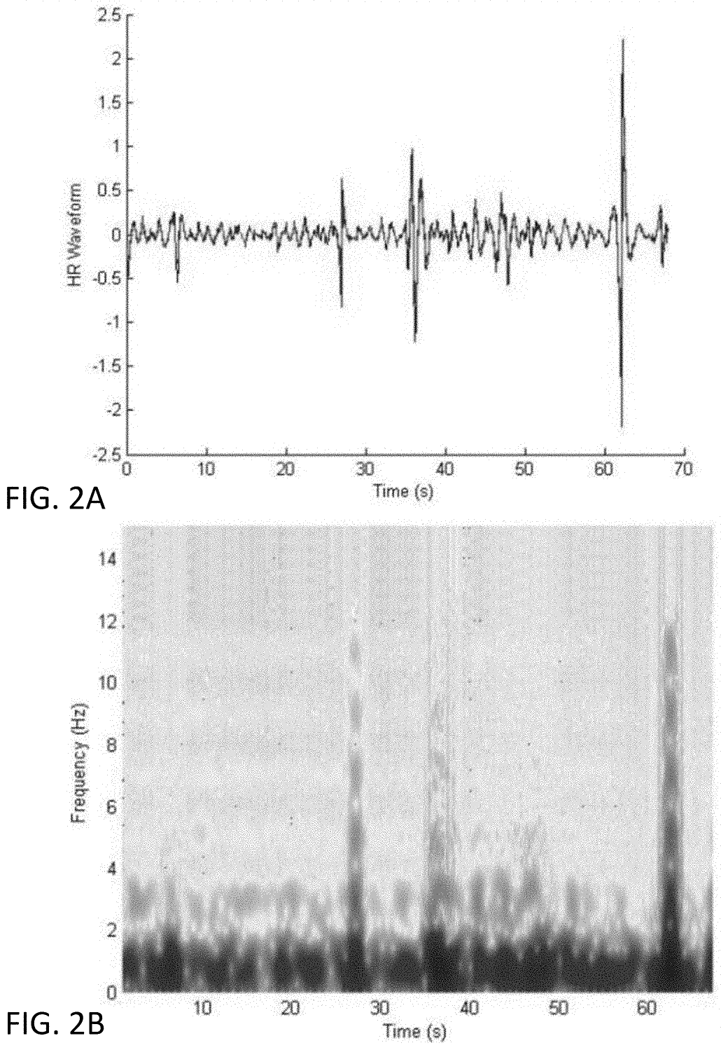

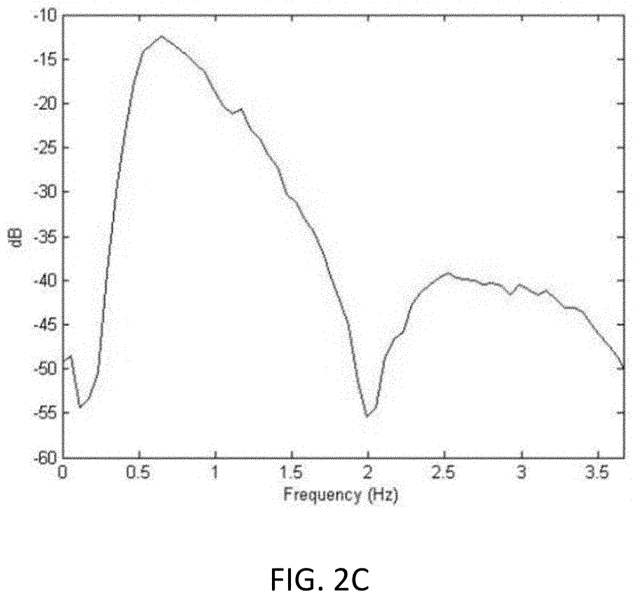

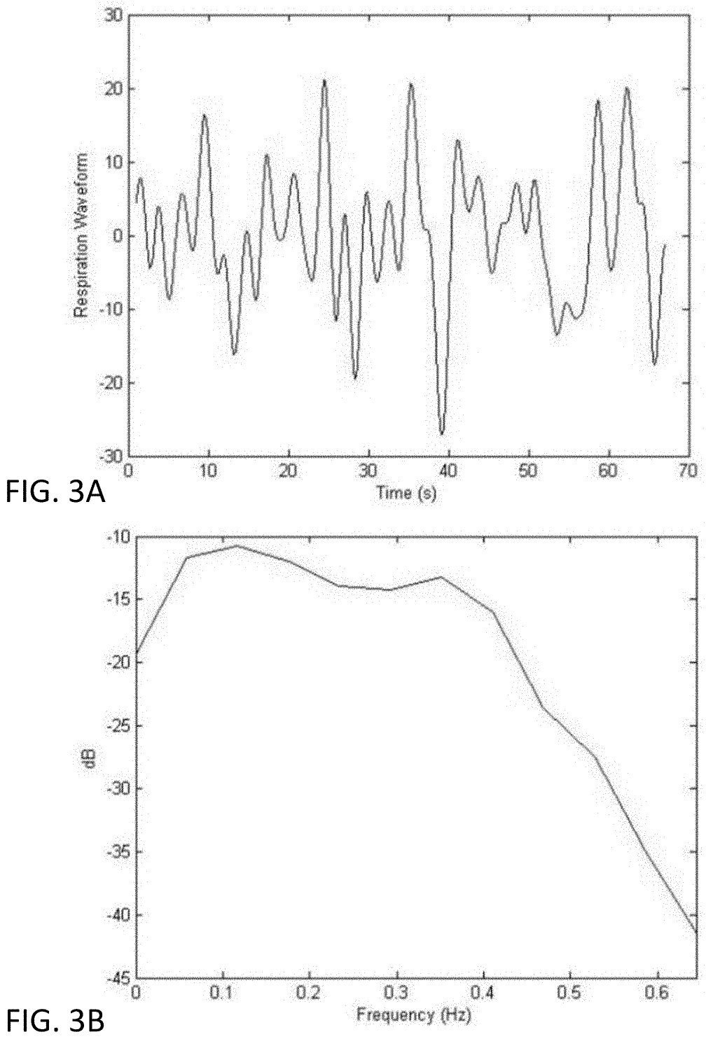

An example implementation of integrating physiological measurements component 112 with a mobile device upon which mobile application 110 is executing is described in more detail below. In this implementation, a video is recorded while a user is holding their finger lightly against the camera lens of a mobile phone and instantaneous heart rate and respiratory rate are estimated based on the recorded video. Each frame of the video comprises an array of pixels (e.g. 480×640), each of which has a specific color hue characterized by red, green, and blue components with integer values ranging from 0 to 255. The red, green, and blue color values are averaged within each frame, and combined across frames to yield an N×3 array of average color hues, where N is the number of frames in the video. Temporal changes in the color of the video frames are analogous to blood passing through the capillaries of the fingertip. FIG. 2A illustrates a projection of the recorded data onto its first principal component. This projection technique maximizes the variance in the recorded time series to highlight any periodic signal content associated with heart rate. The resulting Nx 1 time series is passed through a 4th order, bandpass, Butterworth infinite impulse response (IIR) filter with cutoff frequencies that limit signal content to within a reasonable physiological range for heart rate.

Analysis of the signal in the time or frequency domain enables estimation of the instantaneous heart rate. For example, FIG. 2B shows a spectrogram of the signal, which employs the Fast Fourier Transform (FFT) on a sliding window of data to decompose signal power into frequency bands as a function of time. In the spectrogram, frequency bands with higher power density are shown with darker colors and lower power density are shown with lighter colors. The spectrogram illustrates that the majority of the signal content is in the 0-2 Hz (0-120 cycles per minute) range, and that the dominant frequency component (frequency component with the highest power) changes as a function of time during the recording. Extracting the dominant frequency component for the entire signal, may be achieved, for example, by using Welch's method to estimate the power spectral density. FIG. 2C shows a plot of the power spectrum (signal power in dB as a function of frequency) for the signal data shown in FIG. 2B. Assuming that variations in the time series are primarily due to the pulsatile pumping of blood through the capillaries, the frequency component with maximum power (i.e., the dominant frequency) is the heart rate. In this case, the maximum of the estimated power spectrum occurs at approximately 0.67 Hz, which translates to a heart rate of 40 beats/minute.

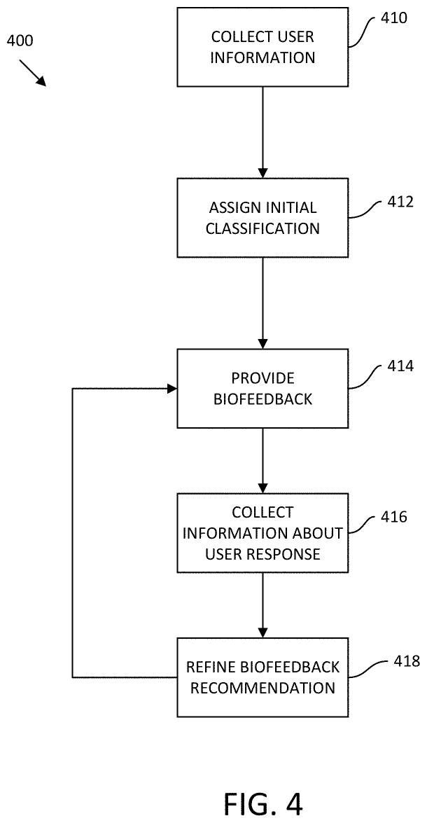

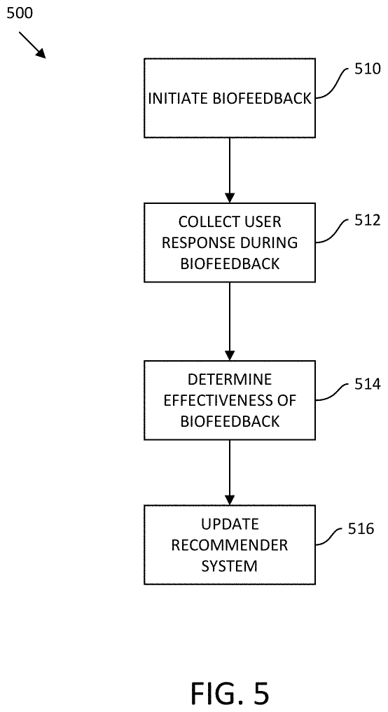

Returning to the spectrogram of FIG. 2B, the dominant frequency, or heart rate, appears to change with time during the recording. Assuming that the subject is at rest, variations in heart rate are likely due to respiration. To isolate this signal, the dominant frequency may be extracted from each window of data used to generate the spectrogram, and bandpass filter using a 4th order Butterworth IIR filter with cutoff frequencies that are physiologically relevant for respiration as shown in FIG. 3A. Welch's method can be used to estimate the power spectral density for this time series as shown in FIG. 3B. Based on the power spectrum, the dominant frequency component of this signal is approximately 0.12 Hz, or around 7 breaths/minute.

It should be appreciated that the signal processing techniques described herein are merely exemplary and other techniques for deriving physiological parameters based on recorded data may alternatively be used. For example, techniques that leverage different types of filtering (e.g., wavelets) and estimation algorithms (e.g., recursive Bayes, Kalman filters) may alternatively be used to determine physiological parameters in accordance with some embodiments.

Additionally, although the example described above records data using a camera of a mobile device, it should be appreciated that physiological data may be recorded using any other suitable type of measurement sensor (e.g., microphones, capacitive touch sensors, etc.), and embodiments are not limited in this respect.

Recommender systems are typically used to provide product or service recommendations based on a user's previous experiences and the experiences of users having similar characteristics. For example, an online retailer may track online behavior to determine which products a user views, and provide products recommendations to the user based on their similarity to the viewed products and also based on products viewed by other users having similar demographic characteristics and/or browsing profiles. In accordance with some embodiments, recommender system 116 is configured to provide personalized biofeedback recommendations that leverage a database of demographic information, usage history, and outcomes from previous users as well as the usage history and changes in objective measures of panic attack severity and duration for the given user.

In some embodiments, recommender system 116 is configured to generate a biofeedback recommendation for a user using a collaborative filtering technique. In collaborative filtering, a recommendation is provided based on other users who have similar characteristics. As shown in FIG. 1, recommender system 116 draws upon information in database 114 that describes characteristics of users of system 100. Initially, database 114 may be seeded with information for characteristics from a plurality of users collected, for example, during their participation in a clinical trial. The characteristics may include demographic information such as gender, socio-economic status, and age, medical history information such as general anxiety level, typical panic attack triggers, and trauma history. Additionally, each of the users may be provided with different types of biofeedback during a panic attack and information related to effectiveness of each type of biofeedback therapy may be recorded in database 114. The different types of biofeedback provided to users may include biofeedback having different modalities (e.g., tactile, audio, visual, or a combination thereof) and/or biofeedback having a same modality (e.g., audio), but having different presentation characteristics (e.g., frequency, amplitude) such that the feedback information is perceived differently by the user for each type of biofeedback.

Effectiveness of a particular biofeedback therapy provided to a user may be quantified in any suitable way, examples of which include, but are not limited to, frequency of the attacks, duration and severity of the attacks, changes in physiological measures (e.g., heart rate, respiratory rate) during an attack, and the user's subjective level of anxiety. As should be appreciated from the foregoing examples, some measures of effectiveness may relate to information captured during a single panic attack whereas other measures may relate to information captured over multiple panic attacks. Information related to one or more of these measures may be recorded in database 114 and groups of users having similar and/or shared characteristics may be represented in the database. For example, the user groups represented in the database 114 may be formed based on the biofeedback approach that most efficiently reduces the duration and/or severity of their panic attacks as characterized by their measured physiological response during the attack. Statistical models for classifying a user into one of the user groups may be trained on feature sets of demographic information using supervised machine learning techniques, examples of which are known in the art.

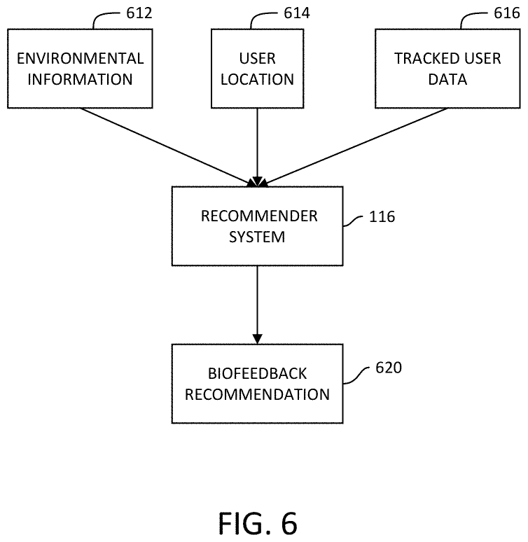

FIG. 4 shows a flowchart of a process 400 for providing personalized biofeedback for a new user of system 100 in accordance with some embodiments. In act 410 information for the new user is collected. The collected information may include demographic information and medical history information, examples of which are described above. Process 400 then proceeds to act 412, where the collected information is used to provide an initial classification of the user into one of several user groups represented in the database 114. Based on the initial classification the recommender system 116 may output a recommended biofeedback type and provide the recommended biofeedback type to mobile application 110. Process 400 then proceeds to act 414, where biofeedback is provided to the new user when the user experiences a panic attack. Process 400 then proceeds to act 416 where information about the user's response to the biofeedback is collected. Process 400 then proceeds to act 418, where the collected information is used to refine a biofeedback recommendation for the user. As the user receives biofeedback therapy for their panic attacks, the success of each feedback approach is quantified, for example, in terms of the severity and duration of the attack. These parameters are compared to previous attacks from that user and this information is used to inform the recommender system in making a recommendation for further personalizing the feedback approach used during the next attack.

The recommendations output from recommender system 116 may be based, at least in part, on existing users who have similar characteristics and have had similar responses to that feedback. When the user experiences an attack, mobile application 110 may be configured to employ the feedback approach suggested by the recommender system. Each time the user has an attack, the recommender system 116 is provided with additional data about the user's response to the biofeedback that enables increasingly accurate suggestions for the type of biofeedback that will be most effective for that particular user.

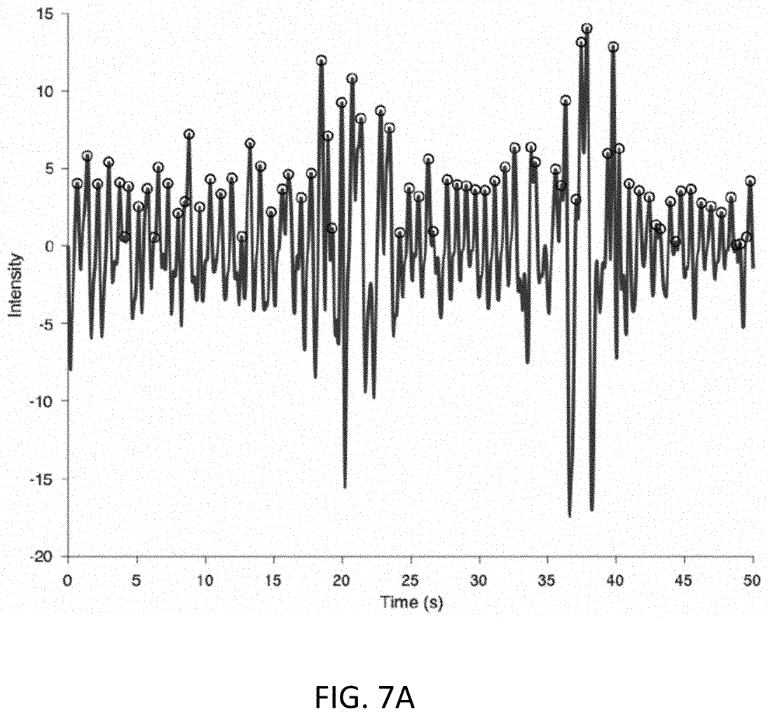

FIG. 5 illustrates a flowchart of a process 500 for providing biofeedback to a user in accordance with some embodiments. In act 510, a biofeedback presentation to the user is initiated. In some embodiments, the user may indicate to mobile application 110 (e.g., via a user interface) that the user is experiencing a panic attack and that biofeedback is desired. In other embodiments, one or more sensors arranged to automatically detect the onset of a panic attack are used to initiate presentation of biofeedback. For example, a heart rate sensor worn by the user may detect an elevated heartrate and this information alone or in combination with other information may be used to detect the onset of a panic attack. In response to determining that biofeedback presentation should be initiated, system 100 is configured to determine the type (e.g., modality and characteristics) of the biofeedback to present. Determination of the type of biofeedback to provide may be made in accordance with a recommendation provided by the recommender system 116, as described above. For example, receipt of a request to initiate biofeedback may cause mobile application 110 to transmit information to recommender system 116 via one or more networks to determine a recommended biofeedback for the user associated with the device on which the mobile application is executing. Additionally or alternatively, information about one or more recommended biofeedback therapies may be stored locally on a mobile device on which mobile application 110 is executing to enable rapid selection of a biofeedback type and/or selection of a biofeedback type when a connection to the recommender system 116 is not available (e.g., when the mobile device does not have an Internet connection).

Following initiation of biofeedback, process 500 proceeds to act 512, where information about the user's response is collected during presentation of the biofeedback and/or following resolution of the attack. Any suitable information may be collected including, but not limited to, the duration of the attack, physiological parameters (e.g., heart rate, respiratory rate) determined directly or indirectly during the attack, a “severity” measure of the attack determined based on data recorded during the attack, and subjective information about the attack provided by the user during and/or following resolution of the attack. Process 500 then proceeds to act 514, where the effectiveness of biofeedback for treating the attack is determined based, at least in part, on the user response information collected during the attack. The effectiveness of biofeedback therapy may be determined in any suitable way. For example, the user response information may be compared to historical user response information for the user. Changes in the user response information over time indicating that the severity and/or frequency of the attacks is decreasing (or increasing) when a particular type of biofeedback is presented to the user may be used to determine a level of effectiveness of the treatment for the user. Process 500 then proceeds to act 516 where the recommender system is updated. For example, the user response information and/or information derived from the user response information (e.g., a generalized “severity” measure) may be provided to recommender system 116 as a new data point for the recommender system to use when recommending future biofeedback for the user. Although shown as two separate acts, in some embodiments, act 514 and act 516 may be combined such that the effectiveness determination is made by the recommender system 116 based on inclusion of the most recent user response information into the database 114.

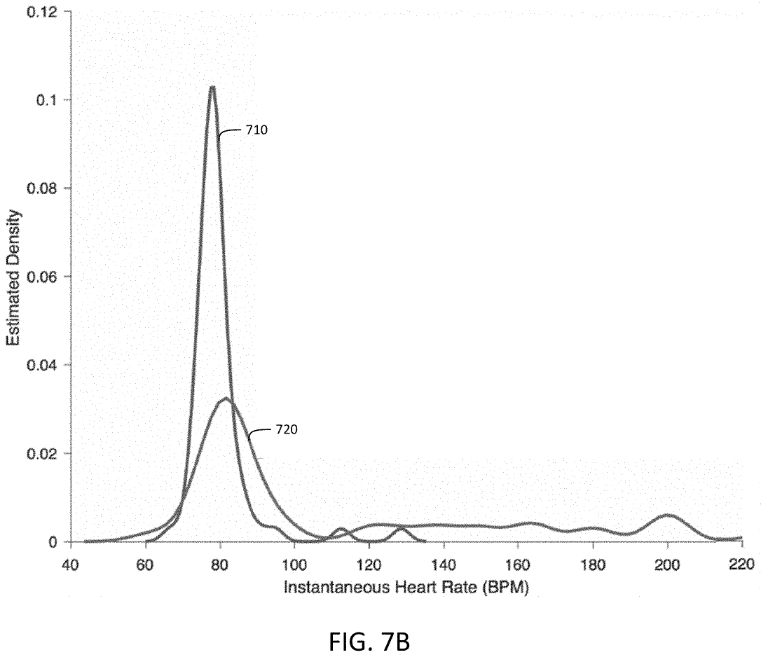

FIG. 6 schematically illustrates a process for determining a biofeedback recommendation by the recommender system 116. In accordance with some embodiments, recommender system 116 includes a plurality of inputs in addition to user response information and information about how similar users responded to different types of biofeedback. For example, as shown, recommender system 116 may be configured to receive as input environmental information 612 (e.g., whether the user is in an environment in which providing particular types of biofeedback is not desired). The environmental information 612 may be determined in any suitable way. For example a microphone associated with the mobile device may be activated to determine a noise level of the user's environment, and audio-based biofeedback may be disfavored when the user is determined to be in a noisy environment. In other embodiments, the user may provide information about environmental information 612, for example, via a user interface provided by mobile application 110. Recommender system 116 may also be configured to receive as input user location information 614. For example, system 100 may include a location determination component (e.g., a global positioning system (GPS) sensor) configured to determine a location of the user, and the recommender system may use information about the user's current location to determine a biofeedback recommendation for the user. Recommender system 116 may also be configured to receive as input tracked user data 616, examples of which include, but are not limited to, the user's current physiological parameters and user activity information (e.g., the user's sleep history, the user's general activity level). Based, at least in part, on the inputs provided to recommender system 116, the recommender system provides a biofeedback recommendation 620 to mobile application 110. The biofeedback recommendation 620 may include both the modality of the biofeedback (e.g., audio, visual, tactile, electrical, or some combination) and characteristics about the biofeedback (e.g., amplitude and/or frequency characteristics of feedback).

Recommender system 116 may be implemented using any suitable architecture that provides as output a personalized biofeedback recommendation for a user. For example, an artificial neural network (ANN) may be used to initially cluster data corresponding to data (e.g., demographic and user response data) collected for a plurality of users to define user groups, and the ANN may be used to classify a new user into one of the user groups to select an initial biofeedback recommendation for the new user. Any suitable classification technique may alternatively be used for classification, examples of which include, but are not limited to, other neural network architectures, support vector machines, and decision trees.

Although shown as a separate component, recommender system 116 may be implemented, at least in part, on the mobile device on which mobile application 110 is executing. In other embodiments, recommender system 116 may be implemented using one or more network-connected (e.g., cloud-based) computing resources communicatively coupled to the mobile device on which mobile application 110 is executing. Such embodiments reduce the computational resources required by the mobile device to perform the biofeedback recommendation. For example, because of the complexity of the calculations required, a recommender system 116 may be implemented using the Surprise Scikit in Python running on an Amazon Web Service (AWS) instance. A REST application programming interface (API) may be used to allow mobile application 110 to send data entered by the user and captured during each attack to database 114. Data may be provided from mobile application 110 to database 114 at any suitable intervals. For example, if the mobile device is connected to the database 114 via a network, the data may be provided as soon as it is available at mobile application 110. In instances in which the mobile device on which the application is executing is not connected to the network, the data may be sent from the mobile application 110 to the database 114 once the mobile device has access to the network. In some embodiments, receipt of new data by database 114 may trigger the recommender system 116 to use the new data to produce an updated biofeedback recommendation for the user. The newly recommended biofeedback approach may be stored by database 114 and may be accessed by application 110 using the REST API when the mobile device has access to the network and/or the recommended biofeedback approach may be transmitted to the application 110 such that the recommendation can be accessed (e.g., from local cache memory) even when the mobile device does not have network access. Once the application retrieves the updated biofeedback strategy for the user, the user application may be reconfigured to enable providing biofeedback in accordance with the recommended approach.

Database 114 may be implemented in any suitable way. As described above, in some embodiments, data is sent to, and retrieved from, the database by mobile application 110 using a REST API. In some embodiments, the database is configured to store user information in an instance of Amazon AWS. The user information may include, but is not limited to, descriptive user information provided by the user (e.g., age, location, panic triggers) and their mobile phone (e.g., user sleep history, user activity history), usage data from mobile application 110 (e.g., recorded or derived physiological information recorded during an attack), the type of biofeedback provided for each attack, details about the feedback implementation for each attack (e.g., volume of auditory feedback, intensity of vibrotactile feedback), instructions for how the mobile application 110 should configure biofeedback for future attacks experienced by the user, push notifications to send to users through the mobile application, contextual information about external groups the user belongs to, and permissions for viewing data from users (e.g., whether a physician is allowed to view data from their patients).

In some embodiments, system 100 also includes a configurable web dashboard 118. The dashboard may be configured to cater to different groups of stakeholders. For example, the dashboard 118 may allow users to visualize the raw physiological data from each panic attack they've tracked using the mobile application 110 as well as statistics about their attacks (e.g., duration and intensity of each attack, time between attacks, their location when attacks occur, attack triggers) and how these statistics have changed over time. Additionally, if approved by the user, a user's physician can view this same information (e.g., or a subset of the information). The user's physician may be able to interact with the dashboard to send push notifications to the user to remind them to track their attacks with the mobile application 110, provide encouragement about their progress, or make suggestions for how they may alter their behavior to reduce the instance of panic attacks. In some embodiments, the push notifications may be sent to database 114 by dashboard 118 using a REST API. When mobile application 110 queries database 114 for an updated biofeedback approach, the database may also extract any stored push notifications scheduled to be relayed to the user. Another group of stakeholders that may be provided access to user information via dashboard 118 are organizations who have an interest in monitoring aggregate data from a group of users rather than individual user data. Such stakeholders may be granted access to a different configuration of the dashboard 118 that provides aggregate information regarding compliance, the prevalence, frequency, and severity of attacks, and the efficacy of this treatment paradigm in various populations.

The techniques described herein relate to providing personalized biofeedback therapy to a patient experiencing a panic attack. However, it should be appreciated that providing biofeedback using a recommender system as described herein may also be used to treat other medical conditions examples of which include, but are not limited to, prevention of falls and treatment of migraine headaches. In such implementations, the biofeedback provided to the user may be tailored to treat the specific medical condition, though the system architecture used in such embodiments would be substantially similar to that described herein. In one such implementation, a system designed in accordance with the techniques described herein is configured to employ cueing to normalize spatio-temporal gait parameters and improve turning and transitions in patients with Parkinson's Disease. In another implementation, a system designed in accordance with the techniques described herein is configured to provide biofeedback that facilitates the management of symptoms associated with multiple sclerosis (MS).

### Illustrative Experiment

A sample of twelve panic attack sufferers (19-34 years old) were studied. To be eligible for the study, subjects had to own a smartphone, have experienced a panic attack in the last two weeks, not be diagnosed with psychosis or schizophrenia, not be opioid dependent, and be able to record video of the fingertip twice daily and whenever a panic attack occurred.

During an initial visit, each of the subjects completed a Structured Clinical Interview (SCID—Panic Module) to quantify the subject's typical panic attack frequency. Subjects were trained to complete the protocol which included collecting a 30-second video of their fingertip twice daily and whenever they had a panic attack in the seven days following the initial visit. After collection of each video, the subject completed an online questionnaire related to the recording and provided the video to study staff.

The questionnaire included items to identify the type of recording (normal daily or panic attack). If the recording was identified as a panic attack, items included questions about ability to record a video of their fingertip, if the recording was difficult to make, and if so what made it difficult. Additionally, the subject was asked to rate the intensity of their panic attack. Finally, the subject was asked if the act of recording the video stopped the panic attack or made it less severe. The data reported by the subjects (written responses to questionnaire and video files) was analyzed for multiple reported panic attacks.

Following the initial visit, nine subjects completed the questionnaire and provided a video at least once (median=8, range=28). In total, the questionnaire was completed 83 times and accompanied by a video 80 times. Compliance issues (attrition of N=11) are common in studies of patients with heightened anxiety, and thus not unexpected in this sample.

Three subjects suffered five panic attacks during the study. Two subjects were able to record a video of the panic attack four out of these five times. In the one instance when the video was not recorded, the subject noted that he “did not remember to record” the video during the attack. The remaining four instances (median intensity 7 of 10) were triggered by pending exams (two attacks) or interpersonal relationships (two attacks). Subjects indicated that it was difficult to make the recordings in three of the four attacks citing external (“other people being around wondering what I was doing”, one attack) and internal factors such that “focusing on something else” besides their feeling of helplessness and worries was difficult (two attacks). Despite subjective reports of difficulty, both users successfully captured and uploaded their videos. One subject was able to record three panic attacks, indicating that it was difficult to make recordings during the first two, but responded that “it wasn't” difficult on the third, possibly suggesting a relatively fast learning curve to video recording panic attacks. Importantly, for three out of the four panic attacks, the subject indicated that the act of recording their fingertip stopped their panic attack. This included positive responses from each of the two subjects. Videos provided daily and during each panic attack allowed analysis of the subject's heart rate. FIG. 7A provides an example filtered average pixel intensity time series during one subject's panic attack. Detected beats are indicated with circles, the timing of which was used to estimate instantaneous heart rate. FIG. 7B provides the distribution of instantaneous heart rates observed during 60-second daily life recordings (trace 710) and panic attack recordings (trace 720).

The results shown in FIGS. 7A-B demonstrate that data can be successfully recorded during a panic attack. For the example data provided, the median heart rate during the reported attack was 8 beats per minute (bpm) higher (86 vs. 78 bpm) than the daily recording taken at the same time of day. While this difference is moderate, it is also worth noting the difference in the distribution of heart rates observed during this time. The daily life recording has a narrow peak in the distribution centered at 78 bpm. In contrast, the panic attack recording has a much wider peak centered at 82 bpm with a long tail indicating observed instantaneous heart rates as high as 200 bpm. This is the panic attack as well as additional artifacts in the recording. According to subject responses from the SCID, the nine users who participated in the daily life portion of the study experienced between three and nine panic attacks each month. However, during the study, only three subjects reported attacks. One user who typically experienced between five and seven attacks each week only experienced three attacks during the study. The reduction in the number of attacks experienced by subjects suggests that there may be a placebo effect associated with this mode of treatment. This phenomenon is similarly observed in placebo-controlled interventions for preventing panic attacks. In three out of the four recorded panic attacks, the subject indicated that the act of recording their fingertip stopped the attack. This is a promising result, especially given that biofeedback was not being applied, and supports the use of mobile biofeedback as a treatment for panic attacks. This result is also supported by cognitive-behavioral theories of panic such that encouraging a panic attack sufferer to approach their symptoms instead of avoid them (i.e. via distraction) is shown to be an active mediator of CBT intervention.

Subjects indicated that it was difficult to record a video of their fingertip during the panic attack. However, results suggest that there may be a learning effect. Specifically, one subject experienced three attacks during the study. They indicated difficulty recording videos for the first two of these attacks, but not for the third. The subject also indicated that the act of recording the video stopped all three attacks. This may suggest that users can learn how to easily make these measurements during their panic attacks, and without impacting the potential efficacy of the treatment modality.

Wrist-worn and other wearable devices are increasingly able to capture heart rate continuously. However, these devices come with additional cost that may be prohibitive to some users. Moreover, as suggested above, it may be that the act of pressing one's finger against the camera lens is important for treatment.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A mobile electronic device configured to provide personalized biofeedback therapy to a user experiencing a panic attack, the mobile electronic device comprising:
at least one computer processor; and
at least one storage device encoded with a plurality of computer-executable instructions that, when executed by the at least one computer processor perform a method comprising:
receiving an indication that the user is experiencing a panic attack;
receiving, from a recommendation system, a recommended biofeedback therapy for the user;
providing the recommended biofeedback therapy to the user during the panic attack; and
transmitting to the recommendation system, information related to the panic attack after the panic attack has subsided, wherein the information related to the panic attack is used to provide an updated recommended biofeedback therapy for the user.

2. The mobile electronic device of claim 1, further comprising:
at least one sensor configured to record physiological data from the user, and
wherein receiving an indication that the user is experiencing a panic attack comprises determining based, at least in part, on the recorded physiological data that the user is experiencing a panic attack.

3. The mobile electronic device of claim 2, wherein the recorded physiological data comprises a heart rate and/or a respiratory rate of the user.

4. The mobile electronic device of claim 2, wherein the at least one sensor comprises an imaging device integrated with the mobile electronic device.

5. The mobile electronic device of claim 4, wherein the recorded physiological data comprises a heart rate and/or a respiratory rate of the user determined from at least one image captured by the imaging device.

6. The mobile electronic device of claim 1, wherein the method further comprises:
tracking, during the panic attack, physiological data associated with the user, and wherein the information related to panic attack includes at least some of the tracked physiological data.

7. The mobile electronic device of claim 6, wherein the method further comprises:
determining based, at least in part, on the tracked physiological data, a severity measure indicating a severity of the panic attack, and wherein the information related to the panic attack comprises the severity measure.

8. The mobile electronic device of claim 6, wherein the method further comprises:
determining based, at least in part, on the tracked physiological data, an effectiveness of the biofeedback therapy provided to the user during the panic attack, and wherein the information related to the panic attack comprises the determined effectiveness of the biofeedback therapy.

9. The mobile electronic device of claim 8, wherein determining an effectiveness of the biofeedback therapy is based, at least in part, on whether the physiological data changes at a certain rate during the panic attack in response to providing the biofeedback therapy.

10. The mobile electronic device of claim 1, further comprising a first stimulator configured to provide biofeedback therapy using a first modality and a second stimulator configured to provide biofeedback therapy using a second modality different from the first modality.

11. The mobile electronic device of claim 1, wherein receiving the recommended biofeedback therapy comprises receiving the recommended biofeedback therapy before receiving the indication that the user is experiencing a panic attack.

12. The mobile electronic device of claim 1, wherein receiving the recommended biofeedback therapy comprises receiving information describing at least one modality to provide the biofeedback therapy to the user and at least one characteristic of biofeedback therapy to be provided.

13. The mobile electronic device of claim 1, wherein the method further comprises:
providing a user interface configured to prompt the user to enter information about an experience of the user prior to and/or during the panic attack; and
receiving the information about the experience of the user via the user interface in response to the prompting, and wherein the information related to the panic attack comprises at least some information about the experience of the user received via the user interface.

14. A method of treating a panic attack using personalized biofeedback therapy provided on a mobile device, the method comprising:
receiving an indication that the user is experiencing a panic attack;
receiving, from a recommendation system, a recommended biofeedback therapy for the user;
providing the recommended biofeedback therapy to the user during the panic attack; and
transmitting to the recommendation system, information related to the panic attack after the panic attack has subsided, wherein the information related to the panic attack is used to provide an updated recommended biofeedback therapy for the user.

15. The method of claim 14, further comprising:
recording physiological data from the user, and
wherein receiving an indication that the user is experiencing a panic attack comprises determining based, at least in part, on the recorded physiological data that the user is experiencing a panic attack.

16. The method of claim 14, further comprising:
tracking, during the panic attack, physiological data associated with the user, and wherein the information related to panic attack includes at least some of the tracked physiological data.

17. The method of claim 16, further comprising:
determining based, at least in part, on the tracked physiological data, a severity measure indicating a severity of the panic attack and/or an effectiveness of the biofeedback therapy provided to the user during the panic attack, and wherein the information related to the panic attack comprises the severity measure and/or the effectiveness of the biofeedback therapy.

18. The method of claim 14, wherein providing biofeedback therapy comprises providing biofeedback therapy using a first stimulator configured to provide biofeedback therapy via a first modality and/or a second stimulator configured to provide biofeedback therapy using a second modality different from the first modality.

19. The method of claim 14, wherein receiving the recommended biofeedback therapy comprises receiving the recommended biofeedback therapy before receiving the indication that the user is experiencing a panic attack.

20. The method of claim 19, wherein receiving the recommended biofeedback therapy comprises receiving information describing at least one modality to provide the biofeedback therapy to the user and at least one characteristic of biofeedback therapy to be provided.
